(19) United States
(12) Patent Application Publication
(10) Pub. No.: US 2020/0030506 A1
(43) Pub. Date: Jan. 30, 2020

(54) BREAST PUMP APPARATUS

(71) Applicant: Clifson Limited, London (GB)

(72) Inventors: Nicholas Paget, London (GB); Matt Batchelor, London (GB); Natalie Vanns, London (GB); Graham Hodge, London (GB)

(21) Appl. No.: 16/337,323

(22) PCT Filed: Sep. 27, 2017
(86) PCT No.: PCT/GB2017/052889

§ 371 Date: Mar. 27, 2019

(30) Foreign Application Priority Data
Sep. 27, 2016 (GB) 1616397.4

(51) Int. Cl. A61M 1/06; A61M 1/00; A47C 7/62

(52) U.S. Cl. CPC A61M 1/062; A61M 1/0031; A61M 1/007; A61M 2210/1007; A47C 7/624; A61M 2205/07; A61M 1/0037

(57) ABSTRACT

A breast pump apparatus, the apparatus comprising at least one collection unit, a pump, the pump comprising a piston, and a chair, wherein the collection unit is in fluid communication with the pump, such that upon actuation the pump creates suction impulses which are transmitted, in use, to the user's breast via the collection unit, thereby expressing milk from the breast into the collection unit, and wherein the pump is connected to the chair, such that, in use, the relative movement of a first portion of the chair relative to the other parts of the chair, manually actuates and drives the piston of the pump.

### FIELD OF THE INVENTION

The invention is a breast pump apparatus, more specifically, the invention is a mechanical breast pump apparatus. More specifically still, the invention is a manually operated mechanical breast pump apparatus. Most specifically, the invention is a chair operated mechanical breast pump apparatus.

### BACKGROUND AND PRIOR ART

Breast feeding has increased in popularity following repeated studies detailing its long and short term health benefits for infants; ‘breast is best’ has been a widespread public campaign. A mother's breast milk contains a number of valuable ingredients including Lactoferrin, secretory IgA, Lysozyme and bifidus factor which are not necessarily present in substitutes and formula. Studies have shown those who were breastfed have a reduced risk of developing later life health problems including diabetes, obesity and asthma. Health benefits also extend to the mother, with studies finding a reduced risk of breast and ovarian cancer. Helping women to breast feed is therefore a driver of considerable health and social benefit and an increase in the number of women breastfeeding is widely viewed as desirable.

One means of facilitating the supply of a mother's milk to an infant is to use a breast pump. A breast pump is used by feeding mothers to extract milk into a collection unit for immediate use or to be stored for later consumption by her child. Breast pumps are often used by mothers whose babies are premature or too sick to breastfeed. In addition, a pre-feed expression of milk allows a mother who is nervous of feeding in public to avoid having to do so.

A considerable proportion of available breast pumps are electric. They have a number of disadvantages. One disadvantage is their loud operating noise which can be a distraction for, or off putting to mothers, which consequently reduces ease of milk expression. For mothers to optimally express milk, reduced stress and minimal environmental distractions are preferred. Amongst other stress factors, new mothers can find the process embarrassing, and this decreases ease of milk expression. There is a widely felt need for a quiet breast pump that is easy to use.

Further, electrical pumps often require mains power to operate, which renders them ineffective when required in areas with no such supply, such as, crucially, disaster zones.

For mothers in developing countries, a manually operated or mechanical pump provides an alternative solution for those without access to electricity. Malnourishment is estimated to cause 45% (3.1 million) of all global child deaths annually (WHO, fact sheet No. 342, January 2016). Where a child is too weak to suckle, the mother's body actually makes it more difficult for the child to extract the milk by increasing the suckling strength required to arouse the breast to express the milk; this is a natural genetic response that encourages ‘survival of the fittest’. The use of a pump can therefore contribute to the avoidance of the deaths of these weaker infants.

Generally, mechanical pumps do not provide the mother with much control over suction pressure. In particular, such pumps often require a complex and longer cessation method involving the machine switch off period and subsequent wait time for the release of the pressure in the suction cup held against the breast during expression. This suction cup, which secures the mouth of the collection unit to the breast; can be a discomfort for feeding mothers. There is a widely felt but relatively rarely articulated need for a breast pump that enables the mother to control and determine their preferred suction pressure and depth of pumping.

Further, there is a need for a pump that reduces irritation and stress, both physical and psychological, to the mother. By allowing control over the suction strength, stroke length and pumping rates, the mother can increase comfort, leading to longer and more regular pumping sessions, with more milk expressed.

It is to these problems amongst others, that the invention attempts to offer a solution.

### SUMMARY OF THE INVENTION

In a first broad, independent aspect, the invention comprises a breast pump apparatus, the apparatus comprising at least one collection unit, a pump, the pump comprising a piston, and a chair, wherein the collection unit is in fluid communication with the pump, such that upon actuation the pump creates suction impulses which are transmitted, in use, to the user's breast via the collection unit, thereby expressing milk from the breast into the collection unit, and wherein the pump is connected to the chair, such that, in use, the relative movement of a first portion of the chair relative to the other parts of the chair, manually actuates and drives the piston of the pump.

The present invention thus enables a negative pressure of around 450 mmHg to be achieved, which is similar to that achieved by larger electrically-driven pumps such as those installed in institutions such as hospitals or the like. However, the present invention achieves this at a fraction of the cost, size and noise levels of those institutional-scale pumps. Furthermore, the present invention allows the mother to control the degree of pumping herself. Yet further, a rocking motion has been indicated as relaxing for mothers.

Advantageously, this pump, provides a dry pump that generates suction by displacement and does not require washing after use. The milk is extracted into, and remains within, the collection unit.

Second, the pump generates suction through piston withdrawal, as operated by the chair's movement. The operator dictates the length of movement an extent to which the piston withdraws from the pump unit, which is particularly advantageous for use in both developed and developing countries, since it provides an effective, non-electric pump apparatus. The ability to alter the piston depth, and suction length, affords the user control over their pumping and can be set for optimal comfort when pumping.

Preferably, the invention comprises a breast pump apparatus, wherein the chair comprises an upper backrest portion and a lower seat portion, and wherein the pump is actuated by the movement of the upper portion relative to the lower portion.

The back portion providing support for the user's back; assisting their user of the chair.

More preferably the invention comprises a breast pump apparatus, wherein the chair is configured such that the pump is actuated and powered by the motion of the user rocking back and forth in the chair.

The rocking motion of expression helps the user, such that she can express milk more easily. The pump is advantageously held in position between the chair portions by straps.

The rocking of the chair back draws and presses the piston respectively, generating suction which is communicated from the pump unit to the collection unit.

Further, the rocking motion requires very little energy input from the user, and little movement to operate, which is beneficial to mothers with restricted food intake.

Preferably the invention comprises a breast pump apparatus, wherein the upper, backrest portion has a first surface, which, in use, supports the user's back, and an opposite, second surface, and wherein the pump has a proximal end and a distal end, the proximal end being attached to the second surface and the distal end engaging, in use, with the floor.

The advantages recited above are embodied in this version of the pump.

Preferably the invention comprises a breast pump apparatus wherein the pump is a syringe pump and wherein the apparatus further comprises a spring also attached to the second surface, said spring being, in use, floor-engaging, and wherein in use, when the user rocks, in use, backwards, the pump is moved from a first, compressed position into a second, extended position and the spring is also moved from a first extended position to a second, compressed position and wherein the resultant opposing force in the compressed spring pushes the backrest in use forward, such that both spring and pump are returned to their respective positions.

Preferably the invention comprises a breast pump apparatus, wherein the upper and lower portions are connected by at least one elongate belt, wherein the piston is attached to an end of the belt and the pump further comprises a spring, and wherein in use, when the user rocks, in use, backwards, the pump is moved from a first, extended position into a second, compressed position and the spring is also moved from a first extended position to a second, compressed position and wherein the resultant opposing force in the compressed spring pulls the backrest in use forward, such that both spring and pump are returned to the first extended position.

This embodiment provides for a breast pump that requires only the floor space necessary for the base portion of the chair. The advantageous positioning of the pumps in suspension on the belts provides ease of control over the pumping depth. The spring to which the piston is connected, acts to aid in the return of the seat back to the starting position, reducing the required exertion of the operator.

Preferably, the invention comprises a breast pump apparatus, wherein there are two belts, driving, in use, two pumps, and wherein each pump is in fluid communication with a separate collection unit.

The advantage of two individual pumps is that they afford the user the capability to adjust each pump to an operator determined optimum suction depth per breast. This feature thereby allows the mother to achieve the ideal suction for each breast, independently. As mothers generally have a dominant feeding breast, it is of benefit to provide an embodiment which compliments those individual requirements as they vary between breasts. Current pumps provide one pump or suction value, which does not accommodate for differing requirements between breasts. Further, the inclusion of two independent pumps provides a breast pump apparatus that can still function even if one pump were to malfunction. Yet further, the provision of two independent pumps maximises the efficiency of each collection unit by provide suitable pressure to both collection units.

Preferably, the invention comprises a breast pump apparatus, wherein the stroke length of the pump is adjustable.

This advantageously provides a means of tailoring suction to an individual user's breast.

Preferably, the invention comprises a breast pump apparatus, wherein the pump further comprises an impeller.

The impeller advantageously provides a method for the ‘feathering’ of the suction, where the resulting effect is to simulate the initial ‘feathering’ suckling of an infant. The initial short burst feathering mimics the natural stimulation for milk expression that a mother's breast is subjected to by a feeding infant's mouth. The impeller can be used by the mother to best replicate the natural suckling pattern of their infant; advantageously providing as close to a natural sensation as is possible and this stimulates an optimal expression of milk.

Preferably, the invention comprises a breast pump apparatus, wherein the chair comprises one or more embedded reinforcing struts at or adjacent the perimeter of the chair. The struts embedded within the base and back support portions of the chair.

The reinforcing struts provide extra structural support, fixed to the centre panel of the chair, preventing loss of the chair shape while providing support for repeated use with minimal reduction in performance. The struts prevent curling inward of the chair portions, which would reduce effectiveness, or result in pump failure.

Preferably, the invention comprises a breast pump apparatus wherein the chair is flat-folding.

The flat folding element of the chair acts to reduce the chairs size and thereby aids with ease of transport. Because it is flat folding, it is also easy to store.

Preferably, the invention comprises a breast pump apparatus, the apparatus comprising at least two collection units, and two manually operated pumps, wherein the collection units are in fluid communication with two manually operated pumps, such that upon actuation, each pump creates suction impulses which are transmitted, in use, to the user's breasts via the collection units, thereby expressing milk from the breasts into the collection unit, the pump further comprising a stroke length controller wherein the stroke length of each pump may be adjusted and set separately from the other pump, such that the suction impulses on each breast may be independently adjusted.

Mothers have a dominant breast and a secondary breast, with each breast responding differently to suction. Advantageously, the pumps can be used in tandem providing two different sorts of suction characteristics if required, or both can be configured to provide the same suction characteristics. The chair can be used with one pump (only) functioning, which provides an advantageous embodiment for a mother who wishes to collect milk from one breast using the pump and feed her infant directly from the other.

In the present invention, the chair may be hinged and may comprise a hinge pump located at a join between a chair back and a base. In such an arrangement, a user may apply a force to the chair back and thus transfer energy to the hinge pump. Such force may be applied by, for example, the user leaning back against the chair back.

A suitable hinge pump may comprise an input shaft for transferring a torque from the join between the chair back and the base to an input shaft, a planetary gear assembly having a sun gear and at least one planet gear, a support ring, a lead screw having a threaded portion on an outer surface thereof, a piston having a threaded portion on an inner surface which corresponds to the threaded portion of the lead screw, and a cylinder head aperture suitable for moving substantially axially within a cylinder bore, wherein a piston seal provides a substantially fluid-tight seal between the piston and cylinder bore. Thus, in use, a torque applied to the input shaft can be translated into movement of the piston along its axis to displace a volume of fluid through the cylinder head aperture. Between the hinge pump and collection unit a hose may be provided to transfer a hydraulic force. Such a hose may be arranged with a fluid-tight connection between the hinge pump and collection unit.

The threaded portions of the respective lead screw and piston could be transposed so that the lead screw has a thread on an inner portion and the piston has a thread on an outer portion.

Such a planetary gear assembly may provide a ratio of 1:4, so that for example a rotation of 10 degrees of the input shaft causes a rotation of 40 degrees of rotation of the lead screw.

A preferred configuration provides a displacement of between around 15 and around 25 ml within the piston, to provide a negative pressure in the collection unit of around 450 mmHg, when the chair back is moved between a first position and a second position. The overall rate of suction is controlled by the rate at which the mother rocks the chair.

Torque may be transferred from the said join to the input shaft by way of a keyway.

Preferably, two hinge pumps are provided, one on either side of the chair of the apparatus of the present invention. The said two hinge pumps are preferably arranged facing one another substantially co-axially with the chair in between their respective cylinder apertures, each pump having opposing lead screw and/or piston thread orientations so that a movement of the chair back relative to the base provides a corresponding displacement in the respective hoses. In other words, in such an arrangement the two hinge pumps work in tandem to drive respective collection units.

One or more of said hinge pumps may be arranged within the hinge of the chair.

Elements of the hinge pump may be formed of plastics material such as polyoxymethylene (sometimes referred to as acetal) or polypropylene for example. The piston seal may comprise Teflon® coating.

The invention also comprises a breast pump apparatus substantially as described herein, with reference to and as illustrated by, any appropriate combination of the text and/or drawings.

### BRIEF DESCRIPTION OF THE FIGURES

The invention will now be described with reference to the figures, of which:

FIG. 1 is a perspective view of the breast pump apparatus chair and pumps of the invention;

FIG. 2 is a cross-sectional side view of the pump of the invention;

FIG. 3 is an elevational side view of the breast pump apparatus in a first position in-use;

FIG. 4 is an elevational side view of the breast pump apparatus in a second position in-use;

FIG. 5 is an elevational frontal view of the invention in use;

FIG. 6 is a perspective side view of the folded invention;

FIG. 7 is a perspective side view of a further embodiment of the invention;

FIG. 8 is a perspective side view of a still further embodiment of the invention;

FIG. 9 is a side view of a hinge pump suitable for inclusion in a hinged embodiment of the apparatus of the present invention;

FIG. 10 is a cross-section of the hinge pump of FIG. 9 taken along line A-A, when the hinge pump is in a extended mode;

FIG. 11 is a further cross-section of the same hinge pump of FIG. 9, when the hinge pump is in a retracted mode;

FIG. 12 is an end view of an input end of the same hinge pump shown in FIG. 9;

FIG. 13 is an end view of an aperture end of the same hinge pump shown in FIG. 9; and

FIG. 14 is a schematic view of a breast pump apparatus exemplifying the present invention.

### DETAILED DESCRIPTION OF THE PREFERRED EMBODIMENTS OF THE INVENTION

Before the present systems and methods are described, it is to be understood that this disclosure is not limited to particular embodiments described, and as such may, of course, vary from them. It is also to be understood that the terminology used herein is for the purpose of describing particular embodiments only, and is not intended to be limiting, since the scope of the present disclosure will be limited only by the appended claims.

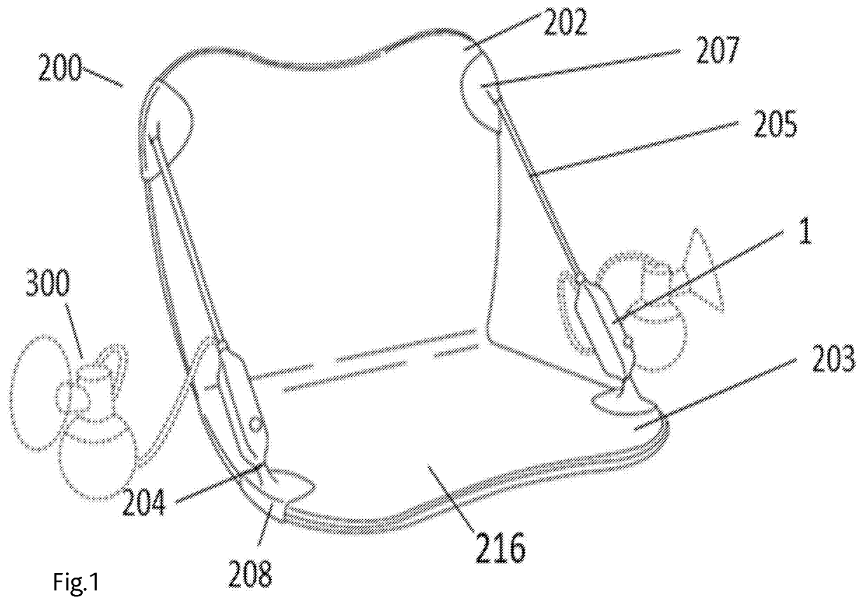

At FIG. 1 the chair 200 comprises a chair back 202 and chair base 203. The pump unit 1 is located between the short strap 204 and long strap 205, both attached to the chair by fasteners 207, 208.

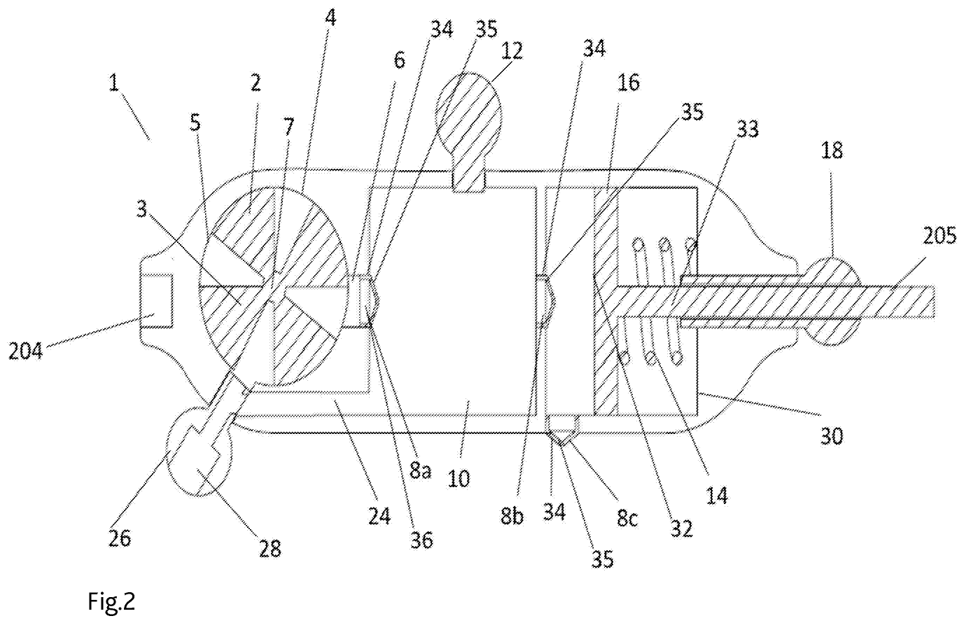

FIG. 2 shows pump unit 1 for a breast pump apparatus, comprising three chambers; the Impeller chamber 4, air reservoir chamber 10 and the piston chamber 30. The pump unit 1 also comprises, an impeller 2, piston 16, non-return valves 8a, 8b, 8c and spring 14. The pump unit 1 housing, comprising a solid plastic or metal construction, provides a case in which the pumping components are contained. The chambers 4, 10, 30 are in this embodiment sized such that they can collect up to 70 mm of air with each stroke of the pump unit 1. Other sizes of pump may be appropriate.

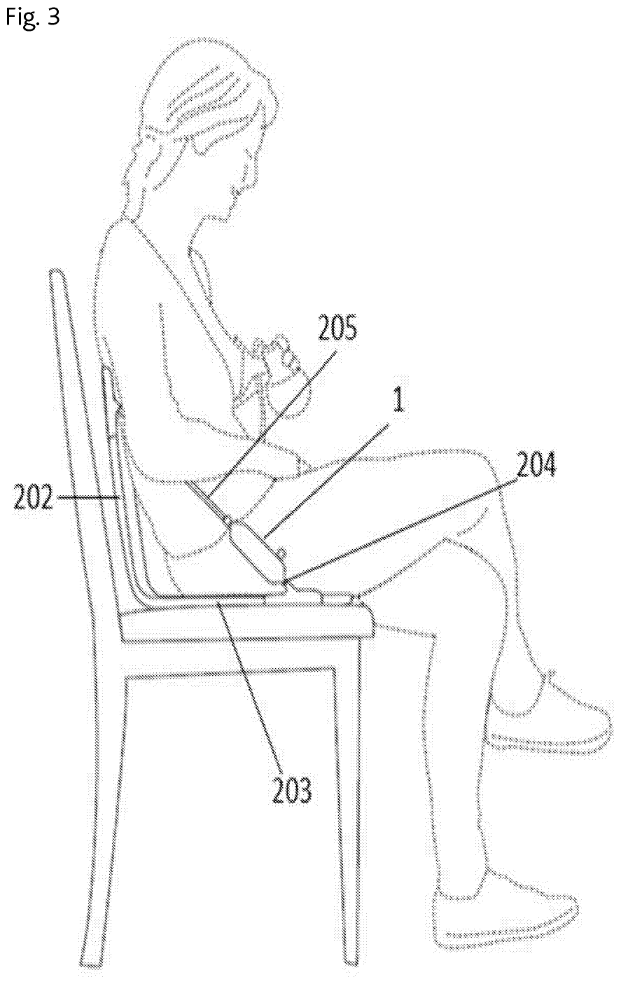

At FIG. 3 it is shown that the pump unit 1 is supported in its position by a connection, which may be permanent, VIA straps 204, 205 that connect to (at FIG. 2) the chair base 203 and chair back 202 portions of the chair. The straps 204, 205 are attached at opposing ends of the pump unit 1, suspending pump unit 1 between the seat portion 203 and back portion 202 of the chair 200. The straps 204, 205 are of a textile, such as nylon composition. Correct material choice provides for a strong strap, durable enough to withstand repeated use, resulting in minimal loss of inherent fabric tension through repeated strain. The strap comprises a longer portion 205 and a shorter portion 204. The longer portion 205 fits into the proximal end of the pump unit 1, complementarily fitting into the fixture so as to form a break-resistant connection, whilst the shorter portion 204 connects with the distal end.

Returning to FIG. 2, the longer portion 205 passes into the pump 1 through the piston adjustment knob 18 to meet the piston 16 of the pump 1. The piston 16 comprises a face 32, preferably of metal, or plastic, so sized and shaped as to provide an airtight seal against the plunger chamber's 30 internal wall, with the injector 33 behind the piston 16 face joining the long strap 205. The shape of the piston chamber 30 is complementary to the piston 16 shape, ensuring a close fit and substantially leak-free operation. The piston spring 14 is positioned behind the face of the piston 16 and encompasses the piston support. The said spring 16 is preferably formed of metal and functions to provide resistance against the piston 16, as well as aiding the piston's return to its starting position, within the chamber 30. The extent of compression of the spring 14 can be adjusted by the piston adjustment knob 18, situated at the long strap 205 end of the pump unit 1. The plunger adjustment knob 18, comprises a helical screw mechanism. The rotation of said piston adjustment knob 18 in a first direction draws more of the strap 205 into the pump unit 1 to lengthen the piston 16 travel and increase the pump's displacement capacity and consequently the amount of subsequent suction generated. It also provides a means of setting the extent of the extension and compression of the spring 16. Rotation of the piston adjustment knob 18 in the opposing direction serves to lengthen the exposure of the shorter strap 205 outside the pump unit 1, which results in the extending of the piston spring 14, drawing the piston 16 towards the end of the pump unit 1 and subsequently decreasing the suction generated by the stroke of the pump when the user rocks backward on the chair.

The piston chamber 30, in front of the piston 16 face, houses two one way, non-return valves 8b, 8c. The non-return valves 8a 8b 8c each comprise a channel 36, onto which a flutter cap 34 is fastened to the opening 35. The cap 34 lifts and returns to the valve opening 35 to intermittently restrict air flow. The piston chamber valve 8b connects the air reservoir chamber 10 to the piston chamber 30. The valve 8b comprises a channel 36 and flutter cap 34 in use, as negative pressure is generated when the piston 16 withdraws, in use, in front of the valve 8b, the flutter cap 34 is pushed towards the piston 16, pressed by the through flow of air which fills the piston chamber 30. This action creates a suction action flowing from the pump unit 1 to the collection unit (not shown). As the piston 16 drives further into the chamber 30 towards the valve 8b, the flutter cap 34 is pressed shut by the force of the air flow. The air within the piston chamber 30 is then forced out through the non-return valve 8c, outside of the pump 1.

The impeller chamber 4, which is, in preferred embodiments located at the opposite end of the pump unit 1 to the piston chamber 30, houses the impeller 2. When, in use, the pump's air supply valve 26 is open, air is fed into the impeller 2 from the air hose connector 28. The impeller 2 is situated about a centre point 7 connection to the pump unit 1. The impeller comprises a plurality of blades 3. The ends of each blade 3 follow the sides of the impeller chamber 4, which is constructed to complement the impeller 2 shape. The complementary shape of the impeller chamber 4 ensures the blade 3 runs close to the chamber 4 walls, or within specifically designed impeller 2 blade running grooves 5. In other words, the radius of the impeller 2 comprises a close match with the internal diameter of the chamber. This results in the space between the blades 3 providing a slot into which air is transported through the chamber 4 and into reservoir chamber 10 by impeller 2 rotation. The slot between each blade 3 constitutes a small pocket for air so transported. As the impeller 2 rotates, the air is fed into the impeller reservoir connecting channel 6, through the non-return valve 8a and then into the air reservoir chamber 10.

The rotation of the impeller 2 is a result of the air flow generated by the piston 16 withdrawing. The piston 16 pull creates a negative pressure in the piston chamber 30 and subsequently draws air through the impeller chamber 4, rotating the impeller 2 in the process, and into the air reservoir chamber 10. The negative pressure draws air from the spaces between impeller 2 blades, in turn rotating the impeller 2. The air leaves the impeller chamber 4 through the non-return valve 8a. This drawing of air into the impeller 2 through the air hose connecter 28 generates suction at the collection unit. The non-return valve 8a prevents air flow entering the impeller chamber 4 from the air reservoir chamber 10, which would otherwise undesirably disrupt the impeller 2 function by providing air flow at opposing points of entry of the chamber 4. The effect of flow through the impeller chamber 4, leads to the shorter, intermittent, suction flow (feathering); this mimics the suckling of an infant at the initial phase of feeding. Control over the regularity at which the feathering occurs and also the lengths of bursts of feathering is exercised by the piston's stroke depth and the pressure reduction valve 12 of the air reservoir chamber 10.

The impeller bypass channel 24 connects the supply hose twist valve 26 to the reservoir chamber 10. Use of the bypass channel 24, alters the suction effect to give a longer, deeper spell of suction for use after the mother's breast has been aroused into milk expression, and mimics an infant's natural suckling pattern, as exhibited during the second phase of suckling. To use the bypass channel 24, the operator closes the supply hose valve 26 to divert air flow away from the impeller chamber 4.

To reduce the suction pressure generated by pump unit 1, the user can utilise the pressure reduction valve 12 of the reservoir chamber 10. Opening the valve 12 creates a direct flow path that enables air pressure neutralisation by drawing air in to the reservoir chamber 10 or expelling air out of the pump unit 1. Closing the air passage, prevents air from the reservoir chamber exiting 10. Partial, fractional opening of the valve reduces the air suction generated by the pump unit 1 at the collection unit, by providing the pump 1 with a second source of air flow.

In use, the user sits in the chair 200, attached collection unit or units to breast or breast and proceeds to operate it. By rocking back on the chair 200 the short strap 204 and pump unit 1 are drawn away from the long strap end 205. More of the long strap 205 is revealed as the piston 16 is drawn towards the piston chamber 30 end, compressing the piston spring 14. The pulling back of the piston 16 creates negative air pressure within the chamber 30 as a result of the chamber's volume increasing without air volume increase, which in turn draws open the non-return valve 8b. Air flows towards the negative pressure from the air reservoir chamber 10 through the non-return valve 8b, filling the piston chamber 30. The flow of air results in suction through the non-return valve 8b through the impeller chamber 4, resulting in impeller 2 rotations to deliver bursts of air and subsequent drawing of air along the air supply hose 28 which in turn generates suction at the collection unit 300. Alternatively, when the impeller chamber 4 is bypassed, the air is drawn into the piston chamber 30 through the air reservoir chamber 10, from the impeller bypass channel 24 which generates suction along the air hose 28 and at the collection unit 300. This suction path generates the longer spells of suction that an infant subjects the mother's breast to once it is feeding.

On the forward rock of the chair 200, the long strap 205 and pump unit 1 move towards the short strap 204. The piston is thereby driven into the chamber 16, towards the non-return valve 8b, which forces the valve 8b shut. The air is expelled through the second non-return valve 8c of the piston chamber 30. As the piston 16 returns, the pump unit 1 generates no suction or ‘blow-back’ at the collection unit as a result of the non-return valve's 8c operation.

At FIG. 1, the chair 200, comprised of a rigid central portion, is equipped with internal support struts 210 (not shown) to prevent loss of shape. These struts 210 are built upon with cushioning 214 (not shown) and an outer layer of durable fabric 216 designed to resist wear from user contact as well as surface and ground contact. The fabric straps 204, 205, which are in preferred embodiments of a textile such as nylon, and are fastened to the chair base 203 and chair back 202 by means connected to the rigid centre of the chair 200; high structural integrity and in particular the rigidity of the central portion, makes pumping easier. The support beams, or struts 210 (not shown), running the length of the internal skeleton of the chair 200, reinforce the chair 200 structure, providing a more durable centre onto which the chair 200 padding is fixed and preventing it from deforming in use.

At FIG. 3, the chair 200, comprising a base 203 and back 202, is shown. The chair 200, whilst in its rocked backward position, pulls the long strap 205 out from pump unit 1.

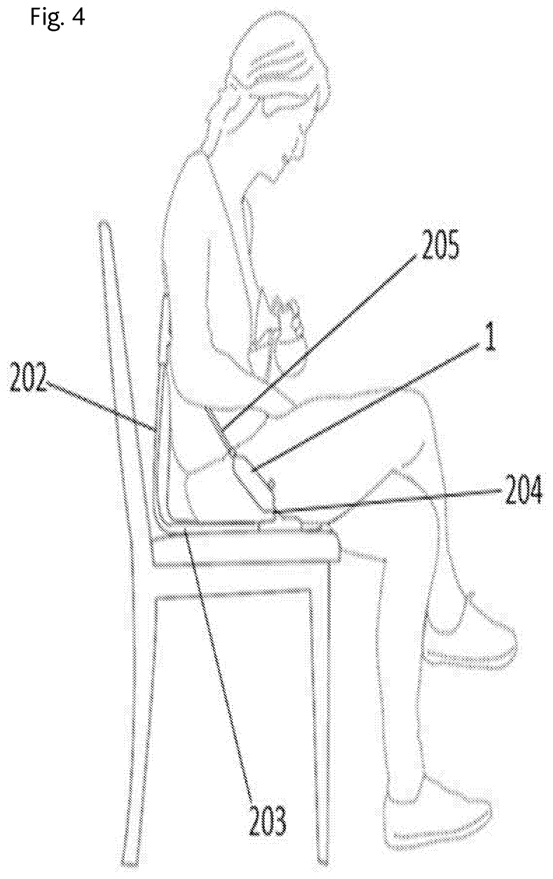

FIG. 4 shows the chair 200 in its rocked forward position, which results in the long strap 205 returning to a first position of lesser exposure, with the piston 16 withdrawn inside the pump unit 1.

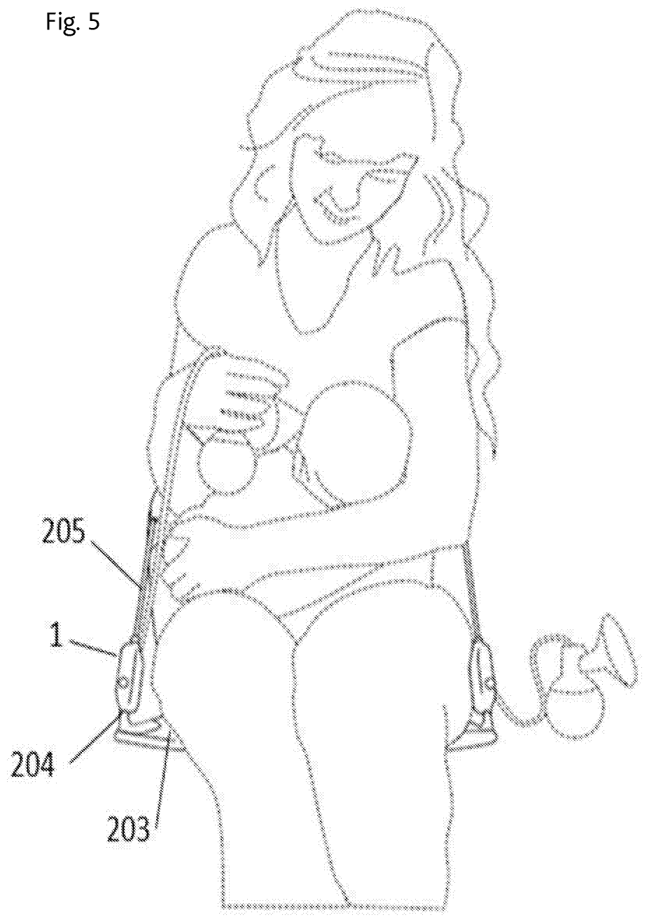

FIG. 5 shows the chair 200 and pump units 1. The pump units 1 are located on either side of the chair 200. By varying the length of the straps 204, 205, the user can alter the depth at which the piston 16 is drawn into the given pump unit 1 and subsequently the pressure generated by a stroke of the pump 1. The chair 200 shape provides a base 203 on which the user sits, to hold the base 203 still. The chair base 203 extends to beyond the gluteal of the user, but does not reach too far as to prevent flexion at the knees, and so as to allow feet to rest upon the floor in a standard seating position. The chair back 202 extends up from the base 203, and is wide enough to allow the user to rock back and forth without the straps 204, 205 or pump unit 1, impeding movement.

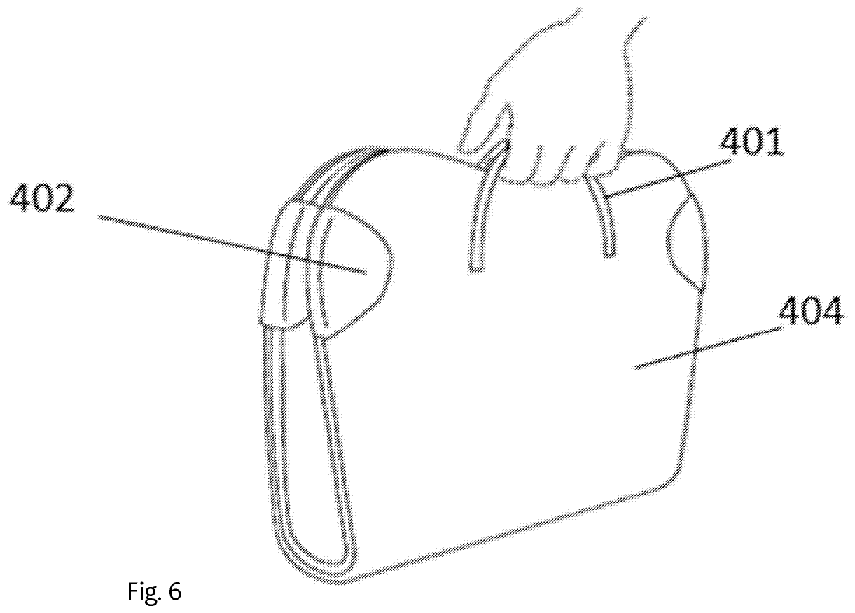

At FIG. 6 the chair base 203 comprises handles 401 in the form of straps 401; these are used as carrying handles. They may preferably be made of nylon or textiles materials. The portion of the chair 200 sat on by the user in use is carried on the inside of the flat-folded chair 200, in this embodiment. The strap fastener 402 wraps around the chair 400 to increase the rigidity of the fastener 402.

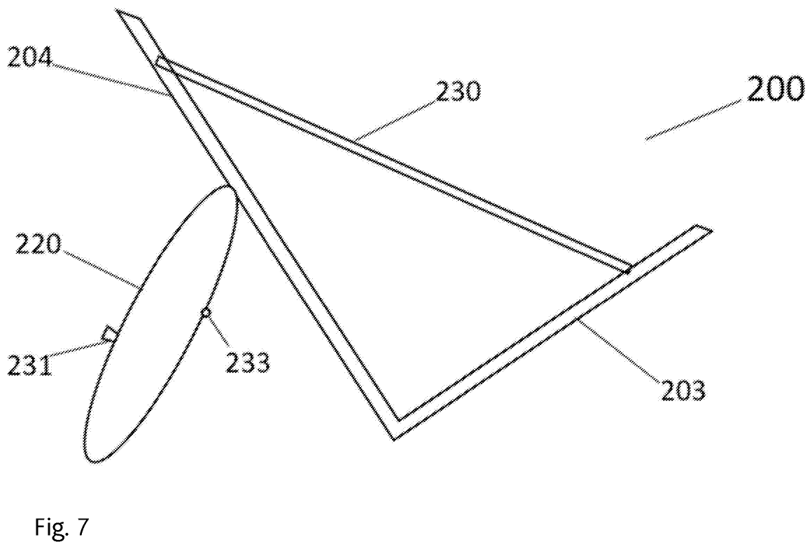

In an alternative embodiment, at FIG. 7, the pump 1 is housed within a leaf spring 220 that affixes to the rear side of the chair back 202 and engages, in use, with the ground. The leaf spring 220 supports the back of the chair 202 by maintaining contact with the ground, and may be compressed in a controlled fashion when the chair 200 is rocked backward. The leaf spring 220, compresses a compressible textile, plastic or metal case that is dimensioned to allow for the changes in the shape (chiefly compression) of the spring 220 during rocking movement. The pumping mechanism remains the same as the previously described pumping unit 1. The rocking back of the chair drives the piston 224 into the piston chamber 225, expelling any air out of the pump 1 via the non-return valve 8c. The forward rock of the chair draws the spring 220 out of its compressed state, the piston 224 is drawn out of the piston chamber 225, drawing air through the air reservoir chamber 227 and through the air hose 229, generating suction at the collection unit 300. The spring 222 aids the user in an analogous returning to an upright position, in the same way as the provision of the straps 204 and 205 in the previous embodiment urged the user back into the first position thanks to the spring 14 in the pump housing and, in certain preferred embodiments, the elasticity of the straps 204 and 205. The spring therefore returns the chair 200 at its initial angle of opening.

In this embodiment, the textile straps 230 are fixed at either end of the chair 200, by a fastening, to the struts 210, thereby maintaining tension. The strap 230 is fastened through the exterior layers, via a reinforced outer wrap 208 that protects the fastening and surrounding fabric join from wear caused by repeated use. The chair 200 remains at a fixed angle to provide the user with the ideal chair angle to operate the leaf spring 220. As in previous embodiments, the pump comprises an air hose connector 231, pressure reduction valve 233 and piston adjustment knob 23. The air hose connector 231 affixes the hose 229 to the pump 1, providing the suction to the collection unit. Rotation of the piston adjustment knob 235 enables the adjustment of suction depth. Rotation of the knob 235 draws the piston 224 toward the piston 224 entrance of the pump 1, compressing the internal piston spring 222 which allows the piston 224 greater span of movement, in turn providing greater suction. Opposite rotation of the piston adjustment knob 235 pushes the piston 224 further into the piston chamber 225, reducing potential piston spring 222 compression, and shortening potential suction depth by providing a smaller, air finable, portion of the chamber 225. Where a smaller volume of space is available, less suction is required through the air hose and collection unit to fill the chamber 225.

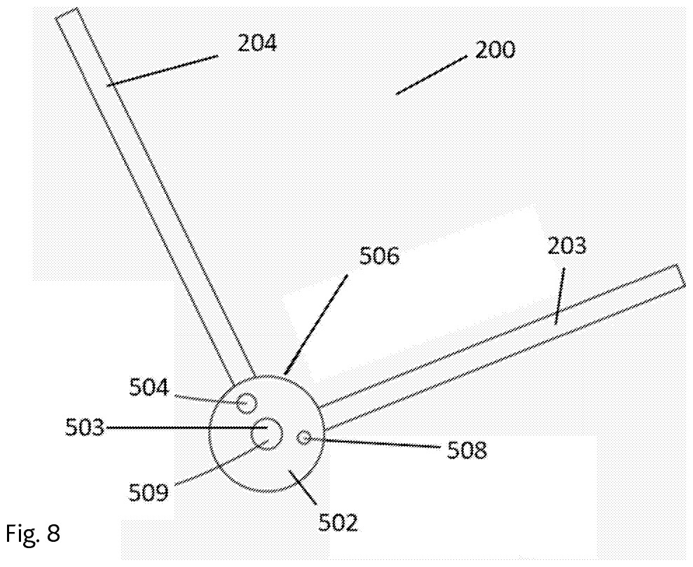

In an alternative embodiment, at FIG. 8, the chair 200 comprises a hinge pump 502 located at the join between chair back 204 and base 203. Hinge pump 502 translates rotational movement of chair back 204 relative to the axis of the hinge 503 into linear movement at the pump, creating negative pressure, generating suction which causes milk to be aspirated from a breast, in use, and drawn away to the collection unit. The internal pumping mechanism can be a cam and follower arrangement, bellows, or an impeller style pump as disclosed in previous embodiments.

The chair back 204 and chair base 203 are fastened to the axis of the hinge 503 of pump 502. As the user leans backwards against chair back 214, the hinge pump 502 is forced open, drawing the internal piston 510 out of the piston chamber 512, and creating negative pressure. The negative pressure generates suction at the collection unit. As the mother rocks forward, the chair back 204, returns to its original upright position, returning the internal piston 510 in to the piston chamber 512, and expelling air from the piston chamber valve 514.

In this embodiment textile straps 230 are not required to provide support between the portions. The hinge pump 502 comprising a return spring, provides resistance to the user asserting backward pressure against chair back 204. The return spring is adjusted by altering the resistance adjustment knob 508 on the hinge pump 502. The return spring resistance may be adjusted relative to the weight and strength preference of the user.

The hinge comprises a pressure adjustment valve 504, allowing the user to adjust the level suction pressure at the collection unit. The chair 200 can comprise more than one hinge pump 502. Analogously to previous embodiments, two hinge pumps 502 can work independently to provide suction to each of the user's breasts. The user can adjust the pressure adjustment valve 504 independently between the pumps to provide the desired suction pressure at each breast.

The chair 200 further comprises a valve for attaching a collection unit 509. The valve for attaching a collection unit 509 is situated on the outer edge of the hinge, reducing interference possibility with the chair back 204 and base 203.

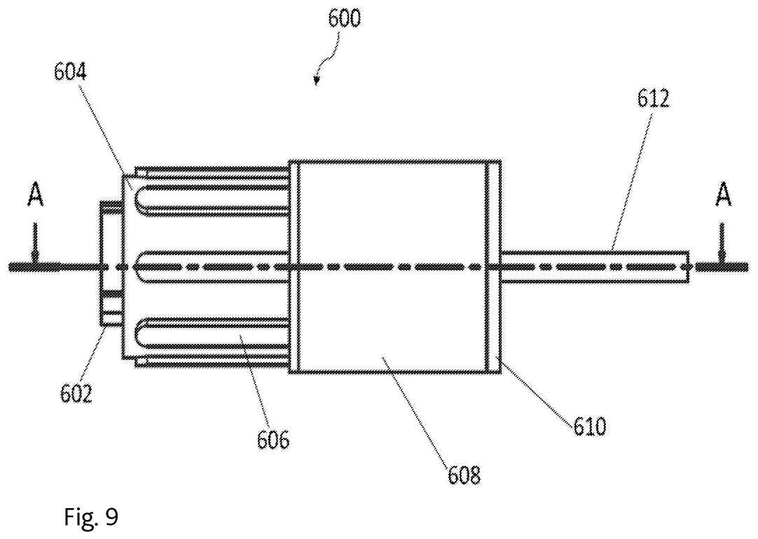

Turning now to FIG. 9, a hinge pump 600 is shown which has an input shaft 602, partially enveloped by and partially extending from a gear box housing 604. An external surface of the gear box housing 604 is provided with a keyway 606 formed by grooves. Adjacent the gear box housing 604 is a cylinder wall 608 which extends towards a cylinder head 610. Extending from the cylinder head 610 is a portion of hose 612.

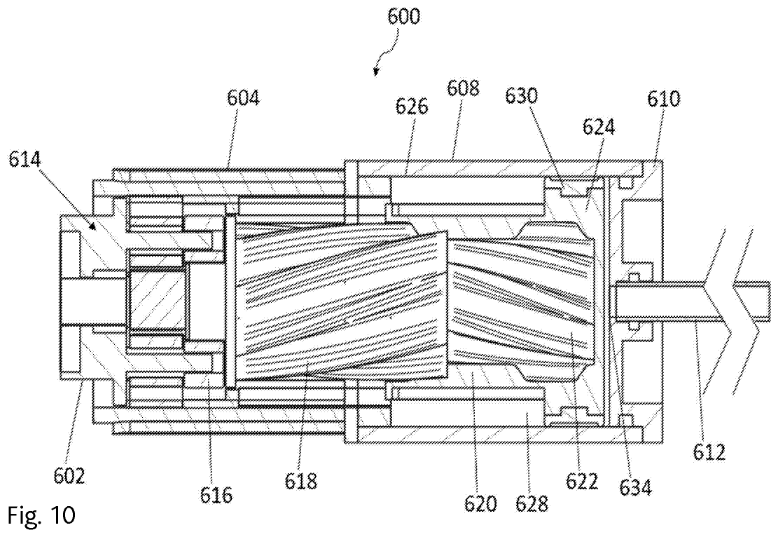

In FIG. 10, the same hinge pump 600 is shown in section along line A-A of FIG. 9. The input shaft 602 is in mechanical connection with a planetary gear assembly 614 which has a support ring 616 which in turn is in mechanical connection with lead screw 618. Input shaft 602 is also in mechanical connection with the gear box housing 604, thus enabling a torque applied to one or both of the input shaft 602 and the gear box housing 604 to be transmitted to lead screw 618 via the planetary gear assembly 614. Lead screw 618 is threaded on its external surface for engagement with a correspondingly threaded inner portion 622 of piston 620. The piston 620 has a head 624 which can move with respect to an internal surface 626 of the cylinder wall 608 which forms a cylinder bore 628. A seal is formed between the head 624 and the cylinder bore 628 by way of a piston seal 630. At one end of the cylinder bore 628 is a cylinder head 610 which generally closes that end of the cylinder bore 628 but which also has a cylinder head aperture 634 which in turn is in fluid connection with a portion of hose 612.

In use, rotation of the lead screw 618 gives rise to an axial movement of the piston 622 within the cylinder bore 628 to extend or retract the piston 622 which, due to the sealed relationship between the piston 622 and the cylinder bore 628 leads to a pressure change in the hose 636 to provide a fluid displacement.

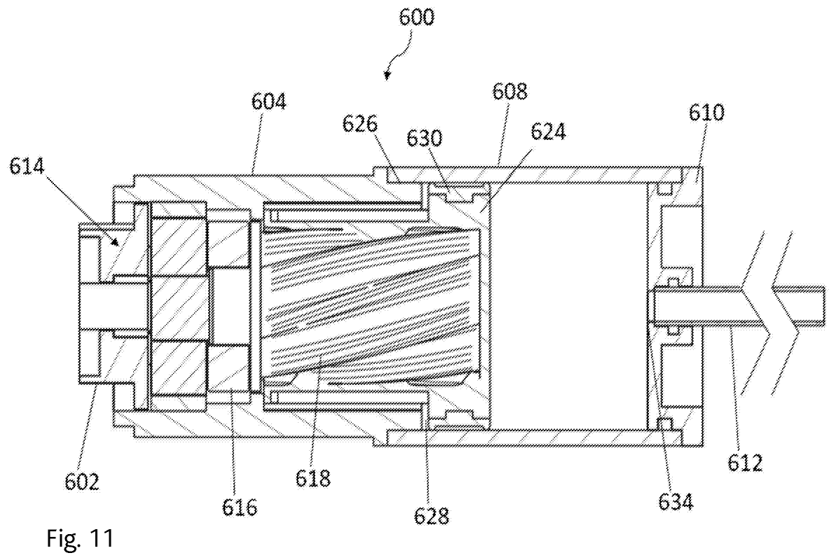

FIG. 11 shows the same hinge pump 600 as shown in FIG. 10, taken along the same section at a rotational different angle. In FIG. 11, the piston 622 is shown in a retracted state thus having a relatively large displacement between the piston head 624 and the cylinder head 632.

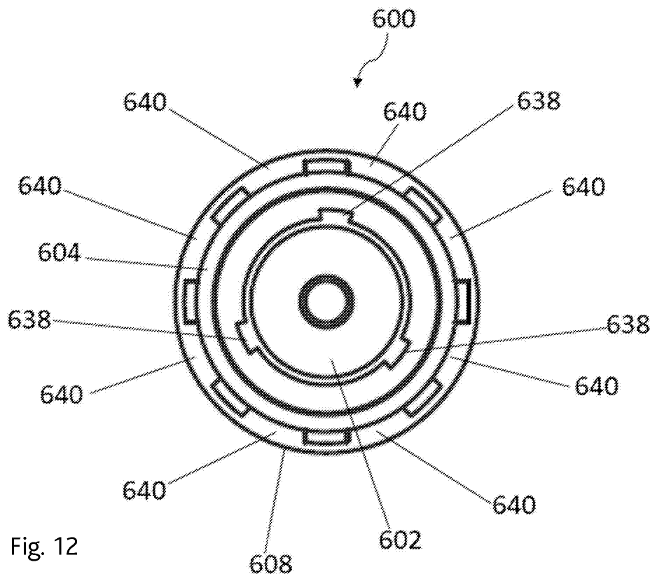

FIG. 12 shows the same hinge pump 600 as viewed from an input end. Input shaft 602 is co-axial with gear box housing 604 and is provided with protrusions 638 for engagement with another corresponding part of the apparatus. Gear box housing 604 is provided with grooves 640 to form a keyway. Cylinder wall 608 has a slightly larger diameter than that of the gear box housing 604.

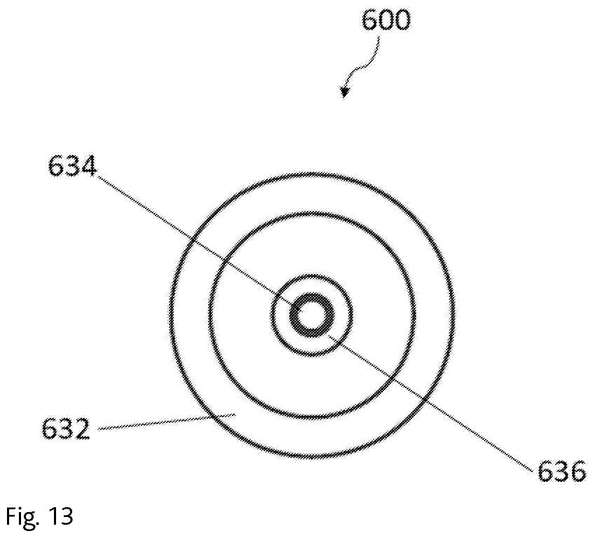

FIG. 13 shows the same hinge pump 600 as viewed from an aperture end. Cylinder head 632 has a cylinder head aperture 634 which is in fluid communication with the cylinder bore (not shown in FIG. 13) and which is connected to a portion of hose 636.

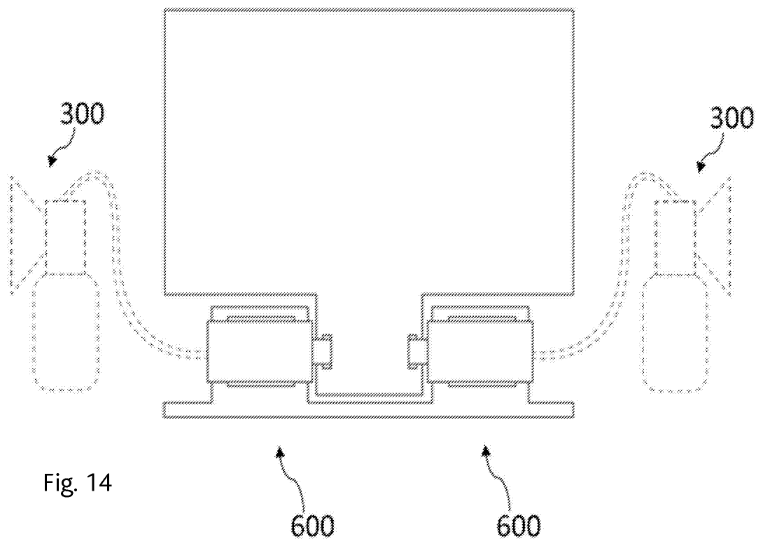

FIG. 14 shows schematically a breast pump apparatus exemplifying the present invention including two hinge pumps 600 as shown in FIGS. 9 to 13 and described with reference to those figures in fluid communication with respective collection units 300.

1. A breast pump apparatus, the apparatus comprising at least one collection unit, a pump, the pump comprising a pumping chamber, and a chair, wherein the collection unit is in fluid communication with the pump, such that upon actuation the pump creates suction impulses which are transmitted, in use, to the user's breast via the collection unit, thereby expressing milk from the breast into the collection unit, and wherein the pump is connected to the chair, such that, in use, the relative movement of a first portion of the chair relative to the other parts of the chair, manually actuates and drives the pumping chamber of the pump.

2. A breast pump apparatus according to claim 1, wherein the pumping chamber comprises at least one of the following; a piston, an impeller and bellows.

3. A breast pump apparatus according to claim 2, wherein the chair comprises an upper backrest portion and a lower seat portion, and wherein the pump is actuated by the movement of the upper portion relative to the lower portion.

4. A breast pump apparatus according to either claim 2 or claim 3, wherein the chair is configured such that the pump is actuated and powered by the motion of the user rocking back and forth in the chair.

5. A breast pump apparatus according to either claim 3 or claim 4, wherein the upper, backrest portion has a first surface, which, in use, supports the user's back, and an opposite, second surface, and wherein the pump has a proximal end and a distal end, the proximal end being attached to the second surface and the distal end engaging, in use, with the floor.

6. A breast pump apparatus according to claim 5 wherein the pump is a syringe pump and wherein the apparatus further comprises a spring also attached to the second surface, said spring being, in use, floor-engaging, and wherein in use, when the user rocks, in use, backwards, the pump is moved from a first, compressed position into a second, extended position and the spring is also moved from a first extended position to a second, compressed position and wherein the resultant opposing force in the compressed spring pushes the backrest in use forward, such that both spring and pump are returned to their respective positions.

7. A breast pump apparatus according to claim 3 or claim 4, wherein the upper and lower portions are connected by at least one elongate belt, wherein the piston is attached to an end of the belt and the pump further comprises a spring, and wherein in use, when the user rocks, in use, backwards, the pump is moved from a first, extended position into a second, compressed position and the spring is also moved from a first extended position to a second, compressed position and wherein the resultant opposing force in the compressed spring pulls the backrest in use forward, such that both spring and pump are returned to the first extended position.

8. A breast pump apparatus according to either claim 6 or claim 7, wherein there are two belts, driving, in use, two pumps, and wherein each pump is in fluid communication with a separate collection unit.

9. A breast pump apparatus according to any of the preceding claims, wherein the stroke length of the pump is adjustable.

10. A breast pump apparatus according to any of the preceding claims, wherein the pump further comprises an impeller.

11. A breast pump apparatus according to any of the preceding claims, wherein the chair comprises one or more embedded reinforcing struts at or adjacent the perimeter of the chair.

12. A breast pump apparatus according to any of the preceding claims wherein the chair is flat-folding.

13. A breast pump apparatus, the apparatus comprising at least two collection units, and two manually operated pumps, wherein the collection units are in fluid communication with two manually operated pumps, such that upon actuation, each pump creates suction impulses which are transmitted, in use, to the user's breasts via the collection units, thereby expressing milk from the breasts into the collection unit, the pump further comprising a stroke length controller wherein the stroke length of each pump may be adjusted and set separately from the other pump, such that the suction impulses on each breast may be independently adjusted.

14. A breast pump apparatus according to any preceding claim, wherein the chair is hinged comprises a hinge pump located at a join between a chair back and a base.

15. A breast pump apparatus according to claim 14, wherein the chair is provided with two hinge pumps.

16. A hinge pump for a breast pump apparatus according to claim 14 or 15, having an input shaft for transferring a torque from the join between the chair back and the base to an input shaft, a planetary gear assembly having a sun gear and at least one planet gear, a support ring, a lead screw having a threaded portion on an outer surface thereof, a piston having a threaded portion on an inner surface which corresponds to the threaded portion of the lead screw, and a cylinder head aperture suitable for moving substantially axially within a cylinder bore, wherein a piston seal provides a substantially fluid-tight seal between the piston and cylinder bore.
